(19) United States
(12) Patent Application Publication
(10) Pub. No.: US 2010/0069323 A1
(43) Pub. Date: Mar. 18, 2010

(54) COMPOSITION FOR APPLICATION TO SKIN OR MUCOSA

(75) Inventors: Tadashi Seto, Osaka (JP); Takayuki Miyano, Osaka (JP); Kenichi Haruna, Osaka (JP); Takahiro Kurose, Osaka (JP)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: ROHTO PHARMACEUTICAL CO., LTD., Osaka-shi, Osaka (JP)

(21) Appl. No.: 12/513,593

(22) PCT Filed: Nov. 9, 2007
(86) PCT No.: PCT/JP07/71830

§ 371 Date: May 5, 2009

(30) Foreign Application Priority Data
Nov. 10, 2006 (JP) 2006-305995

(51) Int. Cl. A61K 31/734; A61P 17/02

(52) U.S. Cl. 514/54

(57) ABSTRACT

Disclosed is a composition for application to skin or mucosa comprising alginic acid and/or salts thereof, which has reduced cytotoxicity against a skin cell or mucosa cell and higher safety, the composition for application to skin or mucosa comprises an alginic acid and/or salts thereof that contains substantially no low-molecular-weight fraction having a molecular weight of 3,500 or less.

### TECHNICAL FIELD

The present invention relates to a composition for application to skin or mucosa that contains alginic acid and/or salts thereof, and that has reduced cytotoxity on skin or mucosal cells. Furthermore, the present invention relates to a method for reducing the cytotoxicity of alginic acid and/or salts thereof.

### BACKGROUND ART

Alginic acid has an action of becoming a gel (more viscous) after being partially cross-linked with a divalent or higher valent cation such as a Ca²⁺ ion; it is already known that alginic acid can be applied as a component for mucosa such as opthalmic mucosa, nasal mucosa and oral mucosa (see Patent Document 1). It has also been discovered that when a formulation containing alginic acid is applied to skin or mucosa, small amounts of Ca²⁺ ions existing on skin or mucosa are contacted with alginic acid and make the formulation gelate (more viscous) on skin or mucosa, and are therefore useful for improving the retention of the formulation on the applied site and maintaining the action produced by active ingredients.

Because skin and mucosa, particularly mucosa, are very sensitive to irritation and toxicity, the components applied to them are required to have low irritation and low toxicity. Concerning the irritation property, it has been reported that alginic acid has not only low-irritation properties, but also the effect of reducing the irritation properties of other compounds (see Patent Document 2). Concerning cytotoxicity, it has been reported that calcium salt of alginic acid exerts cytotoxicity (see Patent Document 3). However, the cytotoxicities of other salts of alginic acid, as well as alginic acid itself, have not been sufficiently examined to date.

Recently, safety has been given increased attention; thus, it is desirable to provide a composition for application to skin or mucosa whose cytotoxicity is reduced as much as possible.

### Patent Document 1: Japanese Patent Application Laid-Open Publication No. 2002-332248

### Patent Document 2: Japanese Patent Application Laid-Open Publication No. 2006-517200

### Patent Document 3: Japanese Patent Application Laid-Open Publication No. 2001-212223

### DISCLOSURE OF THE INVENTION

### Problems to be Solved by the Invention

The object of the present invention is to provide a composition for application to skin or mucosa containing alginic acid and/or salts thereof, which has reduced cytotoxicity to skin or mucosa, as well as increased safety. Furthermore, the object of the present invention is to provide a method for reducing the cytotoxicity of alginic acid and/or salts thereof.

### Means for Solving the Problems

As a result of intensive studies for solving the above-described problems, the present inventors found the following:

(i) As conventional alginic acids and/or salts thereof that are purified from tangle weed and the like, and that are acceptable for living bodies, there are various alginic acids in which the molecular weight ranges from about 380 to 1,000,000. Alginic acid and/or salts thereof comprising a low-molecular-weight fraction having a molecular weight of 3,500 or less exist in about 7 to 20% of them based on a total amount (peak area ratio by GPC method).

(ii) A low-molecular-weight fraction having a molecular weight of 3,500 or less exerts cytotoxicity on mucosal cells; however, a fraction having a molecular weight of more than 3,500 does not exert cytotoxicity on mucosal cells.

(iii) By selecting alginic acid and/or salts thereof that comprises substantially no low-molecular-weight fraction having a molecular weight of 3,500 or less, and mixing them to a composition for application to skin or mucosa, the cytotoxicity of the composition for application to skin or mucosa can be remarkably reduced; therefore, the safety thereof can be increased.

The present invention has been completed by making further improvements based on these findings.

Specifically, the present invention relates to the following compositions for application to skin or mucosa:

Item 1-1: A composition for application to skin or mucosa that contains alginic acid and/or salts thereof comprising substantially no low-molecular-weight fraction having a molecular weight of 3,500 or less.

Item 1-2: The composition for application to skin or mucosa according to Item 1-1, wherein the content of the fraction having a molecular weight of 380 or more to 3,500 or less is 2.5% or less based on a total amount of a fraction having a molecular weight of 380 or more of the alginic acid and/or salts thereof.

Item 1-3: The composition for application to skin or mucosa according to Item 1-1, which contains the alginic acid and/or salts thereof in 0.001 to 20% by weight based on a total weight of the composition.

Item 1-4: The composition for application to skin or mucosa according to Item 1-1, which is a liquid formulation.

Item 1-5: The composition for application to skin or mucosa according to Item 1-1, which is an ophthalmic composition, otologic composition, oral composition, rectal composition, vaginal composition, or urethral composition.

Item 1-6: The composition for application to skin or mucosa according to Item 1-1, which is an eye drop or eye wash.

Furthermore, the present invention relates to the following methods for reducing the cytotoxicity of alginic acid and/or salts thereof:

Item 2-1: A method for reducing the cytotoxicity of alginic acid and/or salts thereof, which comprises the step of removing substantially a low-molecular-weight fraction having a molecular weight of 3,500 or less from the alginic acid and/or salts thereof.

Item 2-2: The method for reducing according to Item 2-1, wherein the step of removing is removing a low-molecular-weight fraction from alginic acid and/or salts thereof so that a fraction having a molecular weight of 380 or more to 3,500 or less is 2.5% or less based on a total amount of a fraction having a molecular weight of 380 or more.

The present invention also relates to the following use:

Item 3-1: Use of alginic acid and/or salts thereof that comprises substantially no low-molecular-weight fraction having a molecular weight of 3,500 or less for the manufacture of a composition for application to skin or mucosa.

Item 3-2: The use according to Item 3-1, wherein the composition for application to skin or mucosa has reduced cytotoxicity.

Item 3-3: The use according to Item 3-1, wherein the content of the fraction having a molecular weight of 380 or more to 3,500 or less is 2.5% or less based on a total amount of a fraction having a molecular weight of 380 or more of the alginic acid and/or salts thereof.

Item 3-4: The use according to Item 3-1, wherein the alginic acid and/or salts thereof is contained in 0.001 to 20% by weight based on a total weight of the composition for application to skin or mucosa.

Item 3-5: The use according to Item 3-1, wherein the composition for application to skin or mucosa is a liquid formulation.

Item 3-6: The use according to Item 3-1, wherein the composition for application to skin or mucosa is an ophthalmic composition, otologic composition, oral composition, rectal composition, vaginal composition, or urethral composition.

Item 3-7: The use according to Item 3-1, wherein the composition for application to skin or mucosa is an eye drop or eye wash.

The present invention also relates to the following use:

Item 4-1: Use of the alginic acid and/or salts thereof that comprises substantially no low-molecular-weight fraction having a molecular weight of 3,500 or less for avoiding the induction of cytotoxicity in a composition for application to skin or mucosa.

Item 4-2: The use according to Item 4-1, wherein the content of the fraction having a molecular weight of 380 or more to 3,500 or less is 2.5% or less based on a total amount of a fraction having a molecular weight of 380 or more of the alginic acid and/or salts thereof.

Item 4-3: The use according to Item 4-1, which is a use for the manufacture of a composition for application to skin or mucosa having reduced cytotoxicity.

Item 4-4: The use according to Item 4-1, wherein the composition for application to skin or mucosa is a liquid formulation.

Item 4-5: The use according to Item 4-1, wherein the composition for application to skin or mucosa is an ophthalmic composition, otologic composition, oral composition, rectal composition, vaginal composition, or urethral composition.

Item 4-6: The use according to Item 4-1, wherein the composition for application to skin or mucosa is an eye drop or eye wash.

The present invention also relates to the following use:

Item 5-1: Use of alginic acid and/or salts thereof that comprises substantially no low-molecular-weight fraction having a molecular weight of 3,500 or less for avoiding the induction of cytotoxicity on skin or mucosa, and increasing the retention of an active ingredient contained in a composition for application to skin or mucosa on skin or mucosa.

Item 5-2: The use according to Item 5-1, wherein the content of the fraction having a molecular weight of 380 or more to 3,500 or less is 2.5% or less based on a total amount of a fraction having a molecular weight of 380 or more of the alginic acid and/or salts thereof.

Item 5-3: The use according to Item 5-1, which is a use for the manufacture of a composition for application to skin or mucosa having reduced cytotoxicity.

Item 5-4: The use according to Item 5-1, wherein the composition for application to skin or mucosa is a liquid formulation.

Item 5-5: The use according to Item 5-1, wherein the composition for application to skin or mucosa is an ophthalmic composition, otologic composition, oral composition, rectal composition, vaginal composition, or urethral composition.

Item 5-6: The use according to Item 5-1, wherein the composition for application to skin or mucosa is an eye drop or eye wash.

The present invention also relates to the following use of alginic acid and/or salts thereof:

Item 6-1: An alginic acid and/or salts thereof that comprises substantially no low-molecular-weight fraction having a molecular weight of 3,500 or less for avoiding the induction of cytotoxicity on skin or mucosa.

Item 6-2: The alginic acid and/or salts thereof according to Item 6-1, which comprises a fraction having a molecular weight of 380 or more to 3,500 or less in an amount of 2.5% or less based on a total amount of a fraction having a molecular weight of 380 or more.

Item 6-3: The alginic acid and/or salts thereof according to Item 6-1, wherein the composition for application to skin or mucosa is a liquid formulation.

Item 6-4: The alginic acid and/or salts thereof according to Item 6-1, wherein the composition for application to skin or mucosa is an ophthalmic composition, otologic composition, oral composition, rectal composition, vaginal composition, or urethral composition.

Item 6-5: The alginic acid and/or salts thereof according to Item 6-1, wherein the composition for application to skin or mucosa is an eye drop or eye wash.

The present invention also relates to the following use of alginic acid and/or salts thereof:

Item 7-1: An alginic acid and/or salts thereof comprising substantially no low-molecular-weight fraction having a molecular weight of 3,500 or less for avoiding the induction of cytotoxicity on skin or mucosa, and increasing the retention of an active ingredient contained in a composition for application to skin or mucosa on skin or mucosa.

Item 7-2: The alginic acid and/or salts thereof according to Item 7-1, which comprises a fraction having a molecular weight of 380 or more to 3,500 or less in an amount of 2.5% or less based on a total amount of a fraction having a molecular weight of 380 or more.

Item 7-3: The alginic acid and/or salts thereof according to Item 7-1, wherein the composition for application to skin or mucosa is a liquid formulation.

Item 7-4: The alginic acid and/or salts thereof according to Item 7-1, wherein the composition for application to skin or mucosa is an ophthalmic composition, otologic composition, oral composition, rectal composition, vaginal composition, or urethral composition.

Item 7-5: The alginic acid and/or salts thereof according to Item 7-1, wherein the composition for application to skin or mucosa is an eye drop or eye wash.

### EFFECTS OF THE INVENTION

In the composition for application to skin or mucosa of the present invention, the cytotoxicity of alginic acid and/or salts thereof is remarkably reduced; thus, it can be safely used for mucosa, which is sensitive to toxicity.

Alginic acid and/or salts thereof that is used conventionally easily precipitate in solutions; thus, this is difficult to formulate. Alginic acid and/or salts thereof used in the present invention can suppress the formation of precipitation. Thus, the composition for application to skin or mucosa of the present invention also has advantage that it is easy to formulate as compared with alginic acid and/or salts thereof that is used conventionally.

Additionally, the composition for application to skin or mucosa of the present invention has an excellent effect of increasing viscosity and enhancing gel strength, by the action of contained alginic acid and/or salts thereof, on contacting with body fluid such as sweat and tear fluid on skin or mucosa. Thus, the composition for application to skin or mucosa of the present invention is useful for avoiding the induction of cytotoxicity on skin or mucosa, increasing the retention of an active ingredient contained in the composition for application to skin or mucosa, and maintaining the action of the active ingredient.

The method for reducing the cytotoxicity of alginic acid and/or salts thereof of the present invention provides alginic acid and/or salts thereof having the reduced cytotoxicity to a mucosal cell or skin cell; therefore, it is useful for providing basic ingredients that are mixed to a composition for application to skin or mucosa having the reduced cytotoxicity and increased safety.

### BEST MODE FOR CARRYING OUT THE INVENTION

### I. Composition for Application to Skin or Mucosa

The alginic acid is a polysaccharide composed of mannuronic acid (that may be simply referred to as “M” hereinafter) and guluronic acid (that may be simply referred to as “G” hereinafter), and is a block copolymer in which homopolymer fractions of mannuronic acid (MM fractions), homopolymer fractions of guluronic acid (GG fractions) and fractions of randomly arranged mannuronic acid and guluronic acid (MG fractions) are randomly bonded.

In the composition for application to skin or mucosa of the present invention, alginic acid or a salt thereof that comprises substantially no low-molecular-weight fraction having a molecular weight of 3,500 or less is used. “Alginic acid that comprises substantially no low-molecular-weight fraction having a molecular weight of 3,500 or less” means that alginic acid in which a low-molecular-weight fraction having a molecular weight of 3,500 or less is removed, and which does not comprise a low-molecular-weight fraction except a fraction having a molecular weight of 3,500 or less that cannot be removed entirely. More specifically, as an example of an “alginic acid that comprises substantially no low-molecular-weight fraction having a molecular weight of 3,500 or less”, the content of an alginic acid having a fraction having a molecular weight or 380 or more to 3,500 or less (total peak area corresponding to a molecular weight of 380 or more to 3,500 or less) based on a total amount of a fraction having a molecular weight of 380 or more (total peak area corresponding to a molecular weight of 380 or more) (which may be simply referred to as a “content of a molecular weight of 3,500 or less” hereinafter) is 2.5% or less, when the GPC method is carried out, is described below.

Based on the standpoint of reducing the toxicity to skin or mucosal cells, the content of a molecular weight of 3,500 or less in the alginic acid used in the composition for application to skin or mucosa of the present invention is preferably 0 to less than 2.0%, more preferably 0 to less than 1.5%, most preferably 0 to less than 1.0%. In other words, the content of a fraction having a molecular weight of more than 3,500 (a high-molecular-weight fraction) in alginic acid used in the present invention is more than 97.5%, preferably 98.0 to 100%, more preferably 98.5 to 100%, most preferably 99.0 to 100%, based on a total amount of a fraction having a molecular weight of 380 or more.

In the present invention, the molecular weight of the alginic acid and the content of a molecular weight of 3,500 or less can be determined by the gel permeation chromatography method (GPC method). The specific conditions are shown in Reference Examples.

For the alginic acid and/or salts thereof used in the present invention, the composition ratio of mannuronic acid to guluronic acid (M/G ratio; molar ratio) is not particularly limited. For example, alginic acid with an M/G ratio ranging from 0.4 to 4.0 is widely used. The smaller the M/G ratio, the easier it is for the composition to begin gelation. Therefore, it is desirable that the M/G ratio be 2.5 or less, preferably 2.0 or less, more preferably 1.6 or less, and most preferably 1.0 or less from the viewpoint of improved retention of pharmacologically active ingredients on application sites. In the present invention, the M/G ratio is a value calculated by dividing alginic acid into block units, fractionating them, and quantifying each of them; the ratio is specifically determined in accordance with the method as described in A. Haug et al., Carbohyd. Res. 32 (1974), p. 217-225.

In the alginic acid and/or salts thereof used in the present invention, the ratio of the MM fraction, the GG fraction and the MG fraction is not particularly limited, and may be appropriately selected depending on the use or form of the composition for application to skin or mucosa.

The salts of the alginic acid used in the present invention are not specifically limited, so long as their forms are salts of alginic acid that comprise substantially no low-molecular-weight fraction having a molecular weight of 3,500 or less, and are pharmacologically or physiologically acceptable. Specific examples of the alginic acid salts include sodium salt, potassium salt, triethanol amine salt, ammonium salt, and the like. The alginic acid salts may be used alone or in any combination of two or more types thereof.

In the composition for application to skin or mucosa of the present invention, a single type of alginic acid and salts thereof may be used, or any combinations of two or more types thereof may be used, so long as it comprises substantially no low-molecular-weight fraction having a molecular weight of 3,500 or less. In particular, alginic acid, sodium alginate and potassium alginate are preferably used in the present invention because they are water-soluble. From the viewpoint of effectively achieving the effect of the present invention, easy preparation and the like, alginic acid is particularly preferable.

Alginic acid and/or salts thereof having the above molecular weight range can be prepared by removing a fraction having a molecular weight of 3,500 or less via gel filtration of available alginic acid and/or salts thereof, or alginic acid and/or salts thereof purified from tangle weed according to a conventional method, and the like. Alternatively, alginic acid and/or salts thereof having the above molecular weight range can be prepared by adding available alginic acid and/or salts thereof, or alginic acid and/or salts thereof purified from tangle weed according to a conventional method to a solution with lower polarity than that of water, and collecting the generated deposit. Specific examples of solutions with lower polarity than that of water include lower alcohols such as methanol and ethanol, organic solvents such as acetone, and liquid mixtures of these and water.

When alginic acid and/or salts thereof used in the present invention are purified from tangle weed, examples of tangle weed used as materials include Lessonia nigrescens from Chile, Lessonia flavicans from Chile, Macrocystis pyrifera from West Coat, Ecklonia maxima from South Africa, Durvillaea potatorum from Tasmanian, Ascophyllum nodosum from North Europe, Laminaria hyperborea from North Europe, Laminaria digitatafrom North Europe, or the like.

In the composition for application to skin or mucosa of the present invention, the mixing ratio of the alginic acid fraction is not particularly limited, and may be appropriately selected depending on the types of applied mucosa, the form of the composition for application to skin or mucosa, and the like. Specifically, the mixing ratio of the alginic acid fraction (weight conversion of alginic acid) is usually 0.001 to 20% by weight, preferably 0.005 to 10% by weight, based on a total amount of the composition for application to skin or mucosa. Specifically, the mixing ratio of the alginic acid fraction (weight conversion of alginic acid) for each use of the composition for application to skin or mucosa is as follows:

When the composition is an ophthalmic composition, it is usually 0.001 to 1% by weight, preferably 0.005 to 1% by weight, more preferably 0.01 to 0.5% by weight, most preferably 0.01 to 0.2% by weight;
When the composition is a nasal composition, it is usually 0.001 to 10% by weight, preferably 0.01 to 5% by weight, more preferably 0.05 to 1% by weight;
When the composition is an oral composition, it is usually 0.001 to 20% by weight, preferably 0.01 to 10% by weight, more preferably 0.01 to 5% by weight;
When the composition is a rectal, vaginal, or urethral composition, it is usually 0.001 to 10% by weight, preferably 0.005 to 5% by weight, more preferably 0.01 to 2% by weight;
When the composition is a dermal composition, it is usually 0.001 to 20% by weight, preferably 0.01 to 10% by weight, more preferably 0.01 to 5% by weight.

The composition for application to skin or mucosa of the present invention may contain active ingredients (pharmacologically active ingredients, physiologically active ingredients or the like) in addition to the above alginic acid and/or salts thereof. Examples of such ingredients include, but are not limited to, decongestants, drugs for modulating ocular muscles, anti-inflammatory drugs or astringents, antiallergic drugs, vitamins, amino acids, antibacterial drugs or disinfectants, saccharides, polymers or their derivatives, cellulose or its derivatives, local anesthetics, anti-glaucoma drugs and anti-cataract drugs. The pharmacologically active ingredients and physiologically active ingredients preferably used in the present invention include the following ingredients:

Decongestants: for example, α-adrenergic drugs such as epinephrine, epinephrine hydrochloride, ephedrine hydrochloride, oxymetazoline hydrochloride, tetrahydrozoline hydrochloride, naphazoline hydrochloride, phenylephrine hydrochloride, methylephedrine hydrochloride, epinephrine bitartrate, naphazoline nitrate or the like, all of which may be either d-form, l-form or dl-form;

Drugs for modulating ocular muscles: for example, cholinesterase inhibitor with active centers similar to that of acetylcholine, such as neostigmine methylsulfate, tropicamide, helenien, atropine sulfate or the like;

Anti-inflammatory drugs or astringents: for example, zinc sulfate, zinc lactate, allantoin, ε-aminocaproic acid, indomethacin, lysozyme chloride, silver nitrate, pranoprofen, sodium azulene sulfonate, dipotassium glycyrrhizinate, diammonium glycyrrhizinate, diclofenac sodium, bromfenac sodium, berberine chloride, berberine sulfate or the like;

Antiallergic drugs: for example, levocabastine, amlexanox, ibudilast, tazanolast, tranilast, isothipendyl, difeterol, triprolidine, tripelennamine, thonzylamine, mebhydroline, fenethazine, oxatomide, suplatast, sodium cromoglycate, pemirolast potassium or the like;

Vitamins: for example, retinol acetate, retinol palmitate, pyridoxine hydrochloride, sodium flavin adenine dinucleotide, pyridoxal phosphate, cyanocobalamin, panthenol, calcium pantothenate, sodium pantothenate, ascorbic acid, tocopherol acetate, tocopheryl nicotinate, tocopheryl succinate, tocopheryl calcium succinate, ubiquinone derivatives or the like;

Amino acids: for example, aminoethylsulfonic acid (taurine), glutamic acid, creatinine, sodium aspartate, potassium aspartate, magnesium aspartate, magnesium and potassium aspartate, glutamic acid, sodium glutamate, magnesium glutamate, ε-aminocaproic acid, glycine, alanine, arginine, lysine, γ-aminobutyric acid, γ-aminovaleric acid, sodium chondroitin sulfate or the like, all of which may be either d-form, l-form or dl-form;

Antibacterial drugs or disinfectants: for example, alkylpolyaminoethylglycine, chloramphenicol, sulfamethoxazole, sulfisoxazole, sulfamethoxazole sodium, sulfisoxazole diethanolamine, sulfisoxazole monoethanolamine, sulfisomezole sodium, sulfisomidine sodium, ofloxacin, norfloxacin, levofloxacin, lomefloxacin hydrochloride, aciclovir or the like;

Saccharides: for example, monosaccharide, disaccharide, and in particular glucose, maltose, trehalose, sucrose, cyclodextrin, xylitol, sorbitol, mannitol or the like;

Polymers or their derivatives: for example, gum arabic, karaya gum, xanthan gum, carob gum, guar gum, guaiac, quince seed, dammar gum, tragacanth, gum benzoin, locust bean gum, casein, agar, dextrin, dextran, carrageenan, gelatin, collagen, pectin, starch, polygalacturonic acid, chitin and its derivatives, chitosan and its derivatives, elastin, heparin, heparinoid, heparin sulfate, heparan sulfate, hyaluronic acid, chondroitin sulfate, ceramide, polyvinyl alcohol (completely or partially saponified), polyvinylpyrrolidone, polyvinylmethacrylate, polyacrylic acid, carboxyvinylpolymer, polyethyleneimine, ribonucleic acid, deoxyribonucleic acid, macrogol and its pharmaceutically acceptable salts or the like;

Cellulose or its derivatives: for example, ethyl cellulose, hydroxyethyl cellulose, hydroxypropyl cellulose, hydroxypropylmethyl cellulose, methyl cellulose, carboxymethyl cellulose, sodium carboxymethyl cellulose, carboxyethyl cellulose, nitrocellulose or the like;

Local anesthetics: for example, procaine hydrochloride, lidocaine hydrochloride or the like;

Anti-glaucoma drugs: for example, isopropyl unoprostone, epinephrine, apraclonidine hydrochloride, carteolol hydrochloride, dipivefrin hydrochloride, dorzolamide hydrochloride, pilocarpine hydrochloride, bunazosin hydrochloride, bupranolol hydrochloride, betaxolol hydrochloride, befunolol hydrochloride, carbachol, levobunolol hydrochloride, epinephrine dipivalate, distigmine bromide, nipradiolol, timolol maleate, latanoprost or the like;

Anti-cataract drugs: for example, glutathione, pirenoxine and sodium 5,12-dihydro azapentacene disulfonate.

The mixing ratios of each ingredient are known in the field of formulations for application to skin, formulations for application to mucosa, and others. Thus, the mixing ratios of the above ingredients in the composition for application to skin or mucosa of the present invention will be appropriately selected depending on the dosage form of said composition for application to skin or mucosa, the types of pharmacologically active ingredients or physiologically active ingredients, and the like. For example, the mixing ratios of pharmacologically active ingredients or physiologically active ingredients can be selected from a range of about 0.0001 to 30% by weight, preferably about 0.001 to 10% by weight based on a total amount of the composition for application to skin or mucosa.

The composition for application to skin or mucosa of the present invention can comprise one kind of component or additive, or more than one kind of component or additive in combination, which are appropriately selected from various components and additives depending upon the use or form of said composition according to conventional means, so long as the effect of the invention is not impaired. Examples of these components or additives include carriers generally used for preparing semi-solid or liquid formulations (aqueous solvents, aqueous or oily bases, etc.), and a variety of additives such as surfactants, preservatives, disinfectants or antibacterial drugs, pH adjusters, tonicity agents, chelating agents, buffering agents and stabilizers.

Examples of typical components used in the composition for application to skin or mucosa of the present invention include, but are not limited to:

Carriers: aqueous solvent such as water and hydrous ethanol;

Surfactants: for example, nonionic surfactants such as polyoxyethylene (hereinafter referred to as POE)-polyoxypropylene (hereinafter referred to as POP) block copolymer (for example, poloxamer 407), POE-POP block copolymer adduct of ethylene diamine (for example, poloxamine), POE sorbitan monooleate, POE hydrogenated castor oil (for example, POE(60) hydrogenated castor oil) and polyoxyl stearate; glycine-type amphoteric surfactant such as alkyldiaminoethylglycine; cationic surfactants such as alkyl quaternary ammonium salt (for example, benzalkonium chloride, benzethonium chloride) or the like, with the figure in parentheses representing the number of added moles;

Preservatives, disinfectants or antibacterial drugs: for example, alkyldiaminoethylglycine hydrochloride, sodium benzoate, ethanol, benzalkonium chloride, benzethonium chloride, chlorhexidine gluconate, chlorobutanol, sorbic acid, potassium sorbate, sodium dehydroacetate, methyl parahydroxybenzoate, ethyl parahydroxybenzoate, propyl parahydroxybenzoate, butyl parahydroxybenzoate, oxyquinoline sulfate, phenethyl alcohol, benzyl alcohol, biguanide (e.g., polyhexamethylene biguanide), Glokill® (Rhodia) or the like;

pH adjusters: for example, hydrochloric acid, sodium hydroxide, potassium hydroxide, calcium hydroxide, magnesium hydroxide, triethanolamine, monoethanolamine, diisopropanolamine, sulfuric acid, polyphosphoric acid, propionic acid, oxalic acid, gluconic acid, fumaric acid, lactic acid, tartaric acid, malic acid, succinic acid, gluconolactone, ammonium acetate or the like;

Tonicity agents: for example, sodium hydrogen sulfite, sodium sulfite, potassium chloride, calcium chloride, sodium chloride, magnesium chloride, potassium acetate, sodium acetate, sodium hydrogen carbonate, sodium carbonate, sodium thiosulfate, magnesium sulfate, disodium hydrogen phosphate, sodium dihydrogen phosphate, potassium dihydrogen phosphate, glycerin, propylene glycol or the like;

Chelating agents, for example, ascorbic acid, tetrasodium edetate, sodium edetate, citric acid or the like;

Buffering agents: for example, citrate buffer, acetate buffer, carbonate buffer, borate buffer, phosphate buffer or the like. More specifically, citric acid, sodium citrate, acetic acid, potassium acetate, sodium acetate, sodium hydrogen carbonate, sodium carbonate, boric acid, borax, phosphoric acid, disodium hydrogen phosphate, sodium dihydrogen phosphate, potassium dihydrogen phosphate or the like;

Stabilizers: for example, dibuthyl hydroxytoluene, trometamol, sodium formaldehyde sulfoxylate (Rongalite), tocopherol, sodium pyrosulfite, monoethanolamine, aluminum monostearate, glyceryl monostearate, or the like.

The dosage form of the composition for application to skin or mucosa of the present invention is not particularly limited. A variety of dosage forms including liquids, suspensions, ointments, creams, gels, lotions, adhesive preparations and sprays, and aerosols can be listed. Among these dosage forms, liquid is preferably used.

The composition for application to skin or mucosa of the present invention can be prepared using appropriate bases or additives depending upon its formulation and so on according to a conventional method.

The application site of the composition for application to skin or mucosa of the present invention may be skin or mucosa, preferably mucosa. The types of mucosa as application site are not particularly limited, and the composition of the present invention can be applied to various mucosa constituting the living body. Specifically, when the composition of the present invention is used as a composition for application to mucosa, it can be used as an ophthalmic composition for application to eye mucosa such as the cornea and conjunctiva; as an otologic composition for application to nasal cavities, aural cavities and so on; as an oral composition for application to the alveolar arch, tongue, lips of the mouth, oral mucosa and so on; a rectal composition for application to the rectum; a vaginal composition for application to the vagina; a urethral composition for application to urinary tract; or others.

Examples of the above ophthalmic composition include eye drops (including ophthalmic drugs, ophthalmic solutions), eye washes (including eye lotions, collyriums), eye ointments, and solutions for wearing contact lenses, and contact lens care products (cleaning solutions, storage solutions, disinfectant solutions, multi-purpose solutions, and the like). The above eye drops and eye washes include eye drops and eyewashes that can be instilled with contacts in place. As used herein, the term contact lens encompasses all types of contact lenses including hard contact lenses (including oxygen-permeable hard contact lenses), soft contact lenses and so on. Examples of the above otologic compositions include nasal drops (including collunariums and nasal solutions), nasal washes (including nasal wash drugs and nasal wash solutions) and ear drops (including ear drugs and ear solutions). Examples of the above oral composition include, in particular, oropharyngeal drugs, mouth washes and the like. Examples of the above rectal compositions include hemorrhoidal agents, suppositories and the like. Examples of the above vaginal compositions include vaginal suppositories and vaginal creams. Examples of the urethral compositions include demulcents and surface anesthetics.

The composition for application to skin or mucosa of the present invention is preferably used, among the above applications, as an ophthalmic composition, more preferably as an eye drop or eye wash. Among mucosa constituting the living body, eye mucosa is known to be one of the mucosa susceptible to the harmful effects of cytotoxicity; however, the composition for application to skin or mucosa of the present invention can also be used for eye mucosa, which is sensitive to such harmful effect of cytotoxicity, with high safety and without subjecting eye mucosa to cytotoxicity. Eye wash is preferable, since it is used in great quantities for washing eye mucosa.

### II. Method for Reducing Cytotoxicity of Alginic Acid and/or Salts Thereof

As described above, alginic acid and/or salts thereof that does not exert cytotoxicity on a mucosal or skin cell can be provided by substantially removing a low-molecular-weight fraction having a molecular weight of 3,500 or less from alginic acid and/or salts thereof. Thus, the present invention further provides, from a different aspect, a method for reducing the cytotoxicity of alginic acid and/or salts thereof, which comprises the step of removing substantially a low-molecular-weight fraction having a molecular weight of 3,500 or less from the alginic acid and/or salts thereof. In the method for reducing, means for removing substantially a low-molecular-weight fraction having a molecular weight of 3,500 or less from alginic acid and/or salts thereof is described in the above section “I. Composition for application to skin or mucosa”.

### III. Use of Alginic Acid and/or Salts Thereof that Comprises Substantially No Low-Molecular-Weight Fraction Having a Molecular Weight of 3,500 or Less

As described above, the alginic acid and/or salts thereof that comprises substantially no low-molecular-weight fraction having a molecular weight of 3,500 or less is useful for the manufacture of the composition for application to skin or mucosa, particularly the composition for application to skin or mucosa having reduced cytotoxicity. Thus, the present invention further provides the use of alginic acid and/or salts thereof that comprises substantially no low-molecular-weight fraction having a molecular weight of 3,500 or less for the manufacture of a composition for application to skin or mucosa.

Additionally, the alginic acid and/or salts thereof can be used to avoid the induction of the cytotoxicity in the composition for application to skin or mucosa that contains alginic acid and/or salts thereof comprising a low-molecular-weight fraction having a molecular weight of 3,500 or less. Thus, the present invention further provides use of alginic acid and/or salts thereof that comprises substantially no low-molecular-weight fraction having a molecular weight of 3,500 or less for avoiding the induction of cytotoxicity. The present invention also provides the use of alginic acid and/or salts thereof that comprises substantially no low-molecular-weight fraction having a molecular weight of 3,500 or less for avoiding the induction of cytotoxicity on skin or mucosa, and increasing the retention of an active ingredient contained in a composition for application to skin or mucosa.

### IV. Use of Alginic Acid and/or Salts Thereof that Comprises Substantially No Low-Molecular-Weight Fraction Having a Molecular Weight of 3,500 or Less

As described above, alginic acid and/or salts thereof that comprises substantially no low-molecular-weight fraction having a molecular weight of 3,500 or less can be used to avoid the induction of cytotoxicity on skin or mucosa by alginic acid and/or salts thereof comprising a low-molecular-weight fraction having a molecular weight of 3,500 or less. Thus, the present invention further provides alginic acid and/or salts thereof that comprises substantially no low-molecular-weight fraction having a molecular weight of 3,500 or less for avoiding the induction of cytotoxicity on skin or mucosa.

Additionally, the alginic acid and/or salts thereof can be used to avoid the induction of cytotoxicity on skin or mucosa, and increase the retention of an active ingredient contained in a composition for application to skin or mucosa on skin or mucosa. Thus, the present invention further provides alginic acid and/or salts thereof that comprises substantially no low-molecular-weight fraction having a molecular weight of 3,500 or less for avoiding the induction of cytotoxicity on skin or mucosa, and increasing the retention of an active ingredient contained in a composition for application to skin or mucosa on skin or mucosa.

### EXAMPLES

The following Examples and Test Examples illustrate the present invention in more detail, but are not to be construed to limit the scope thereof.

### Reference Example

### Method for Determining the Molecular Weight of Alginic Acid

According to the following method, the molecular weight of alginic acid and the ratio of a molecular weight of 3,500 or less were determined. At first, eight kinds of pullulan standards with known molecular weights were used as molecular weight markers to prepare a calibration curve by plotting the retention times by the GPC method versus molecular weights of pullulan. Then, alginic acid (a corrective alginic acid sample) was subjected to the GPC method, and assumed weight average molecular weight of alginic acid (the corrective alginic acid sample) was obtained according to the calibration curve previously prepared. Furthermore, alginic acid (a corrective alginic acid sample) was subjected to the GPC method where a multi-angle laser light scattering detector (MALS) was used as a detector to give the weight average molecular weight, with which the relational equation previously obtained with pullulan is corrected. Subsequently, by using this corrected relational equation, the molecular weight of the alginic acid and the ratio of a molecular weight of 3,500 or less are determined. The details are as follows.

(1) Measurement Condition of GPC

The gel permeation chromatography method (GPC method) with liquid chromatograph is conducted using the following condition for determination.

(1-1) Detector: differential refractive index detector or multi-angle laser light scattering detector (MALS)
(1-1-1) Multi-angle laser light scattering detector (MALS): DAWN-EOS type multi-angle laser light scattering detector is used.
(1-1-2) Wavelength is set to 690 nm (semiconductor laser).
(1-1-3) Second virial coefficient (A2)×concentration (c) is set to 0 mol/g.
(1-1-4) Refractive index increment (dn/dc) is set to 0.114 mL/g.

### (1-1-5) Temperature is set to 23±2° C.

(1-2) Column: three stainless columns each filled with hydrophilic vinyl polymers having 10,000,000, 400,000 and 5,000 molecular weight exclusion limits for aqueous solutions of polyethylene oxide or polyethylene glycol (particle sizes are 13 μm, 10 μm, 7 μm, respectively) are connected and used. Each stainless column has an inner diameter of 7.8 mm and a length of 30 cm.
(1-3) Column temperature is set to 35° C.
(1-4) Mobile phase: a solution that was obtained by dissolving 11.5 g of ammonium dihydrogen phosphate and 17.5 g of sodium chloride in distilled water to a total volume of 1000 mL and adjusting the pH to 4.0 with phosphoric acid is used.
(1-5) Flow rate of mobile phase is set to 0.75 mL/min. In this study, the stabilization of a base line is important; therefore, temperature control and instrument equilibration must be sufficiently performed to obtain a stable base line. After the mobile phase is run for more than one night with the reference path open, the mobile phase is run again for more than one night with the reference path closed. During analysis, the room temperature should not be changed; the temperature inside the instruments, such as columns, tubes and detectors, must also be kept constant.

(2) Preparation of Calibration Curve with Molecular Weight Marker

Pullulan standards that are available as molecular weight markers (more than eight kinds of pullulan having known molecular weights in a range from 5,900 to 788,000) and glucose are dissolved respectively in the mobile phase described in the above measurement condition of GPC to a concentration of 0.1 w/v %, and each 100 μL thereof is subjected to the GPC method described below to obtain their elution times. A calibration curve for pullulan standards is prepared by plotting the logarithm of a molecular weight M of each pullulan standard (log₁₀ M) on the y-axis and its elution time on the x-axis, determining the linear equation of the calibration curve (Equation 1 below).

y=ax+b Equation 1
y: log₁₀ M
x: elution time

(3) Correction of Liner Equation for Calibration Curve

### (3-1) Preparation of Corrective Alginic Acid Sample

100 g of dried tangle weed (Lessonia nigrescens) is cut, washed with water, and immersed in 3 L of 0.2% by weight of sulfuric acid for 2 hours. The extractive is removed, and the residue is immersed in 1 L of aqueous solution of sodium hypochlorite (available chlorine: 10.5% by weight) for 10 minutes, after which the residue is collected followed by an addition of purified water to 3 L and allowed to stand for 10 hours. The same volume of methanol-water solution acidified with sulfuric acid (2% by weight of sulfuric acid, 90% by weight of methanol, water for the rest) is added to this solution, and the resulting precipitate is recovered by filtration and dried. 3 g of this dried precipitate is added by portions to 250 mL of water, and stirred for 10 minutes. After confirming the homogeneous dispersion in water, 0.825 g of sodium carbonate is added, and the mixture is stirred for another 20 minutes to dissolve the precipitate. After confirming that the precipitate is completely dissolved, water is added to a total weight of 300 g to produce a crude solution of alginic acid. To 200 g of the crude solution of alginic acid, 1,800 mL of acetone is added by portions followed by stirring for 30 minutes, after which the solution is filtered by filter paper (φ110 mm). The residue obtained on the filter paper is collected, and then, by drying it at room temperature, acetone is distilled away to obtain the corrective alginic acid sample.

(3-2) Determination of Assumed Weight Average Molecular Weight of Corrective Alginic Acid Sample with Pullulan Standard

The corrective alginic acid sample prepared above is weighed and dissolved to a concentration of 0.1 w/v % in the mobile phase described in the above measurement condition of GPC to prepare a sample solution. This sample solution is subjected to the GPC method for analysis. In the data processing after the analysis of this corrective alginic acid sample, the peak of alginic acid is divided into i pieces for every 0.1 minutes of elution time, and areas for each section (An) are calculated by an automatic integration method. Additionally, the elution times of each section are assigned to the equation 1 to calculate their M values, which are regarded as the assumed molecular weights of the alginic acid of each section (Mn assumption). Further, the area values for each section (An) are divided by the assumed molecular weight of the alginic acid of its section (Mn assumption) to calculate the assumed number of alginic acid molecules of its section (Nn assumption). The assumed molecular weight (Mn assumption) and the assumed number of molecules (Nn assumption) thus calculated are assigned to the following equation 2 to calculate the assumed weight average molecular weight of the corrective alginic acid sample (Mw assumption).

$${Mw}_{assumption} = {\sum }_{n = 1}^{i} ( \begin{matrix}\begin{matrix}{N}_{n assumption} \times \\ {M}_{n assumption} \times \end{matrix} \\ {M}_{n assumption}\end{matrix} ) / {\sum }_{n = 1}^{i} ( \begin{matrix}{N}_{n assumption} \times \\ {M}_{n assumption}\end{matrix} ) \qquad Equation 2$$

### (3-3) Determination of Real Weight Average Molecular Weight of Corrective Alginic Acid Sample

The corrective alginic acid sample is weighed and dissolved to a concentration of 0.5 w/v % in the mobile phase described in the above measurement condition of GPC to prepare a sample solution. 100 μL of this sample solution is subjected to the GPC method for which a multi-angle laser light scattering detector (MALS) is connected as a detector, and the real weight average molecular weight (Mw real) is determined by the GPC-MALS method. The calculation of the weight average molecular weight is performed by Berry plot analysis for the detected single peak and reading the value of the intercept.

### (3-4) Correction for Linear Equation

By correcting the assumed molecular weight of alginic acid (Mn assumption) so that the assumed weight average molecular weight of the alginic acid (Mw assumption) calculated with Equation 2 is equal to the real weight average molecular weight (Mw real) calculated in the above (3-3) GPC-MALS method, the real molecular weights of alginic acid for each section (Mn real) is determined. Specifically, b' is calculated so that the Mw real is equal to the value calculated by assigning the following values to the Mn assumption and Nn assumption in the above equation 2.

Mn assumption: 10^{ax}^{ₙ}^{+b′}
Nn assumption: An/10^{ax}^{ₙ}^{+b′}

The following equation 3, in which the a and b' obtained above are included, is used for a real calibration curve of alginic acid.

y=ax+b′ Equation 3
y: log₁₀ M_{a1g}
x: elution time
M_{alg}: molecular weight of alginic acid

(4) Calculation of Molecular Weight and Peak Area of Molecular Weight of 3,500 or Less

For alginic acid and/or salts thereof that are targets for determination, the elution time of each peak in the chromatograph obtained by the GPC method is applied to the calibration curve expressed in the above equation 3, and thereby molecular weights of each peak are calculated.

In the present invention, peak areas in the chromatograph obtained by the GPC method are calculated by an automatic integration method using an Agilent 1100 series Agilent ChemStation (Agilent Technologies) with the following setting condition or by a similar method.

Initial Settings

### Slope Sensitivity 5

### Peak Width 10

### Area Reject 100

### High Reject 10

Shoulder prop

Program

0.1 second: Integration OFF
Elution time corresponding to a molecular weight of 1,100,000 of alginic acid: Baseline at Valleys ON
Elution time corresponding to a molecular weight of 1,000,000 of alginic acid: Integration ON
Elution time corresponding to a molecular weight of 3,500 of alginic acid: Split peak ON
Elution time corresponding to a molecular weight of 3,000 of alginic acid: Slope Sensitivity 2.5.

The peak area of the alginic acid with a molecular weight of 3,500 or less is calculated as the peak area of an alginic acid with a molecular weight of 380 or more to 3,500 or less.

### Test Example 1

### 1. Preparation of Alginic Acid

Preparation of Alginic Acid in which a Low-Molecular-Weight Fraction was not Removed

To 5 g piece of dried tangle weed (Lessonia nigrescens) from Chile was added 100 mL of water; the mixture was allowed to stand at room temperature for 60 minutes, followed by heating in a water bath at 90 to 100° C. for 30 minutes while being occasionally stirred with a glass rod. After that, the mixture was cooled at 4° C. for about 3 hours, and filtered using filter paper (ADVANTEC, No. 2, φ110 mm), after which the filtrate was collected. To 50 mL of this solution was added 450 mL of acetone, followed by stirring and filtrating by filter paper in the same way, after which the precipitate was collected. The precipitate was dried to produce alginic acid (Comparative Example 1).

Preparation of Alginic Acid in which Low-Molecular-Weight Fraction was Removed and Low-Molecular-Weight Fraction of Alginic Acid

100 g of dried tangle weed (Lessonia nigrescens) from Chile was cut, washed with water, and immersed in 3 L of 0.2% by weight of sulfuric acid for 2 hours. The exudate was removed, and the residue was immersed in 1 L of an aqueous solution of sodium hypochlorite (available chlorine: 10.5% by weight) for 10 minutes, after which the residue was collected followed by the addition of purified water to 3 L, and allowed to stand for 10 hours. To this solution, the same volume of methanol-water solution acidified with sulfuric acid (2% by weight of sulfuric acid, 90% by weight of methanol, water for the rest) was added, and the resulting precipitate was recovered by filtration and dried. 3 g of this dried precipitate was added by portions to 250 mL of water and stirred for 10 minutes. After confirming the homogeneous dispersion in water, 0.825 g of sodium carbonate was added, and the mixture was stirred for another 20 minutes to dissolve the precipitate. After confirming that the precipitate was completely dissolved, water was added to a total weight of 300 g to produce a crude solution of alginic acid. To 200 g of the crude solution of alginic acid thus obtained was added 1,800 mL of acetone by portions followed by stirring for 30 minutes, after which the solution was filtered by filter paper (ADVANTEC, No. 2, φ110 mm). The residue obtained on the filter paper was collected, and then, by drying it at room temperature, acetone was distilled away to obtain an alginic acid fraction which was not dissolved in acetone (i.e., alginic acid in which a low-molecular-weight fraction was removed; Example 1). Conversely, filtrate obtained after the filtration was collected and acetone and water were distilled away under reduced pressure (100 to 150 mmHg, 25° C.) to obtain an alginic acid fraction which was dissolved in acetone (i.e., a low-molecular-weight fraction of alginic acid; Comparative Example 2).

### 2. Measurement of Molecular Weight Distribution of Prepared Alginic Acid

By using a liquid chromatograph (Agilent 1100 system (Agilent Technologies), Controller/Data processing device: G2170AJ ChemStation, Pump: G1311A, Injector: G1329A, detector: differential refractive index detector G1362A (preset temperature: 35° C.), thermostatic chamber: G1316A) and according to the condition and method described in the above Reference Example, the ratios of a molecular weight of 3,500 or less in the alginic acid obtained in the above Example 1 and Comparative Example 1 and 2 were determined.

The ratio of a molecular weight of 3,500 or less was determined according to the method described in the above Reference Example by preparing the corrective alginic acid sample, determining the weight average molecular weight of the corrective alginic acid sample, preparing a calibration curve with pullulan standards (Shodex P-82, Showa Denko), calculating the molecular weights of each peak and area of said peaks detected by GPC method for the alginic acid of Example 1 and Comparative Example 1 and 2.

The result of the ratios of a molecular weight of 3,500 or less in each alginic acid (Example 1 and Comparative Example 1 and 2) are as follows.

TABLE 1

| | | Comparative | Comparative |
|---|---|---|---|
| | Example 1 | Example 1 | Example 2 |
| Ratio of molecular | 2.3% | 7.7% | 12.4% |
| weight of 3,500 or | | | |
| less | | | |

### 3. Evaluation of Cytotoxicity of Prepared Alginic Acid

The test for toxicity of the alginic acid prepared above (Example 1 and Comparative Examples 1 and 2) to cells was carried out. First, an alginic acid solution was prepared by dissolving each alginic acid to a concentration of 0.2 or 2.0% (w/v) in a cell culture medium of medium 199 (GIBCO) supplemented with 10% (v/v) fetal bovine serum (Daiichi Kagaku Yakuhin). Then, rabbit corneal epithelial cell line SIRC (ATCC number: CCL-60) was plated at 2.0×10⁵ cells/well in 48-well microtiter plate (Corning), and alginic acid solution prepared above was added to obtain a final concentration of 0.1 or 1.0% (w/v) of alginic acid, and the cells were subsequently cultured under the condition of 37° C., 5% CO₂ and 90% relative humidity. To the well without alginic acid solution prepared as a control, was added the same volume of cell culture medium. At 48 hours after the initiation of culture, the supernatant was collected and 10% (v/v) of Cell Counting Kit-8 (Dojin Kagaku) of a reagent for viable cell detection was added, after which it was incubated under the condition of 37° C., 5% CO₂ and 90% relative humidity for 1 hour. After 1 hour, the absorbance (450 nm) of the dye developed by reacting with the viable cells was measured using a spectrophotometer (Thermo Electron). With the observed value of the absorbance, cell viability was calculated in the following equation, and the cytotoxicity of each alginic acid was evaluated.

Cell viability(%)=100×(absorbance of the group in which each alginic acid solution was added−absorbance of the medium containing a reagent for viable cell detection)/(absorbance of the control group−absorbance of the medium containing a reagent for viable cell detection) Equation 5

The results are shown in FIG. 1. By adding 1.0% (w/v) alginic acid solution of Comparative Example 2 was added, high cytotoxicity, specifically cell viability of about 5%, was developed. Moreover, microscopic observation also demonstrated that cells to which alginic acid of Comparative Example 2 were added lost their adhesive capacity and floated. Conversely, it was recognized that the toxicity of the alginic acid of Example 1 is clearly reduced as compared with the alginic acid of Comparative Examples 1 and 2. These results revealed that the cytotoxicity of alginic acid results from the component contained in a low-molecular-weight fraction. Furthermore, it was confirmed that the cytotoxicity to mucosal cells can be reduced by selectively adding the alginic acid that comprises substantially no low-molecular-weight fraction to the composition for application to skin or mucosa.

### Test Example 2

### Evaluation of Irritation of Alginic Acid

An eye drop of the following Formulation Example 1 was prepared using the alginic acid of Example 1. The eye drop was aseptically filled in a plastic container after filtration through a membrane filter with 0.22 μm pore size.

### Formulation Example 1

| Alginic acid of Example 1 | 0.1 | g |
|---|---|---|
| Sodium chloride | 0.44 | g |
| Potassium chloride | 0.08 | g |
| Boric acid | 0.3 | g |
| Borax | 0.035 | g |
| Hydrochloric acid | q.s. | |
| Sodium hydroxide | q.s. | |
| Purified water | q.s. | |
| Total volume | 100 | mL (pH 7) |

This eye drop was administered to the eyes of five healthy adult humans (three males, two females), and the sense of irritation was evaluated according to the following criterion.

### <Method of Evaluation

+: with irritation
±: with mild irritation
−: without irritation

The results obtained are shown in Table 2. The results demonstrated that the eye drop of Formulation Example 1 provided a good sense of use; no subject complained of irritation immediately after the ocular instillation, or even 5 minutes or 10 minutes after the ocular instillation.

TABLE 2

| | Result of evaluation | | |
|---|---|---|---|
| | for irritation (No. | | |
| | of subjects) | | |
| | − | ± | + |
| Irritation immediately after ocular | 5 | 0 | 0 |
| instillation | | | |
| Irritation after 5 min. of ocular | 5 | 0 | 0 |
| instillation | | | |
| Irritation after 10 min of ocular | 5 | 0 | 0 |
| instillation | | | |

The eye drop (Comparative Formulation Example 1), which was prepared in the same manner of Formulation Example 1 except that the alginic acid of Comparative Example 1 was used instead of the alginic acid of Example 1, was evaluated for its irritation after the ocular instillation. The results showed that some subjects reported irritation immediately after the ocular instillation of the eye drop of Comparative Formulation Example 1.

From the above results, it was confirmed that alginic acid comprising a low-molecular-weight fraction having a molecular weight of 3,500 or less exerts the cytotoxicity to mucosal cells, and furthermore, can induce irritation at the time of ocular instillation. In contrast, it was revealed that alginic acid in which a low-molecular-weight fraction having a molecular weight of 3,500 or less was substantially removed has significantly reduced cytotoxicity to mucosal cells, and does not cause irritation at the time of ocular instillation; therefore, it is superior in safety and sense of use.

### Test Example 3

### Evaluation of Irritation by Alginic Acid

An eye drop (Formulation Example 2) in which the concentration of the alginic acid of Example 1 was 0.01 g/100 mL and an eye drop (Formulation Example 3) in which the concentration of the alginic acid of Example 1 was 0.2 g/100 mL were prepared. For the eye drops of Formulation Examples 2 and 3, the components contained were the same as those of Formulation Example 1, except for alginic acid and the concentrations thereof.

The eye drops of Formulation Examples 2 and 3 were evaluated for their irritation after the ocular instillation in the same manner as the above Test Example 2. The result was that no subject complained of irritation with either eye drop of Formulation Example 2 or 3 at immediately after the ocular instillation, or even 5 minutes or 10 minutes after the ocular instillation.

### Formulation Example

The alginic acid of Example 1 was used to prepare the formulations shown in Tables 3 to 5 (Formulation Examples 4 to 11).

TABLE 3

| | Formulation | Formulation | Formulation | Formulation |
|---|---|---|---|---|
| Component | Example 4 | Example 5 | Example 6 | Example 7 |
| (Unit: g/100 mL) | Eye drop | Eye drop | Eye wash | Nasal drop |
| Alginic acid of | 0.1 | 0.01 | 0.05 | 0.2 |
| Example 1 | | | | |
| Tetrahydrozoline | 0.05 | — | — | — |
| hydrochloride | | | | |
| Naphazoline | — | — | — | 0.05 |
| hydrochloride | | | | |
| Dipotassium | — | — | 0.025 | — |
| glycyrrhizinate | | | | |
| Chlorpheniramine maleate | 0.03 | — | 0.003 | 0.5 |
| Pyridoxine | 0.1 | — | — | — |
| hydrochloride | | | | |
| Potassium | — | — | 0.1 | — |
| aspartate | | | | |
| Sodium chloride | — | 0.44 | — | 0.67 |
| Potassium chloride | — | 0.08 | — | — |
| Boric acid | 1.8 | 1 | 1.7 | — |
| Borax | 0.2 | 0.2 | 0.1 | — |
| Sodium | — | — | — | 0.4 |
| hydrogenphosphate | | | | |
| Crystal sodium | — | — | — | 0.9 |
| dihydrogen phosphate | | | | |
| Sodium edetate | — | — | 0.005 | — |
| Polysorbate 80 | — | — | 0.1 | — |
| Hydrochloric acid | q.s. | q.s. | q.s. | q.s. |
| Sodium hydroxide | q.s. | q.s. | q.s. | q.s. |
| Purified water | q.s. | q.s. | q.s. | q.s. |
| pH | 6 | 7 | 6.7 | 6 |

TABLE 4

| | Formulation | Formulation |
|---|---|---|
| | Example 8 | Example 9 |
| Component | Disinfectant for | Oropharyngeal |
| (Unit: g/100 mL) | contact lens | drug |
| Alginic acid of | 0.1 | 0.05 |
| Example 1 | | |
| Sodium azulene | — | 0.02 |
| sulfonate | | |
| Potassium chloride | 0.100 | — |
| Sodium chloride | 0.650 | — |
| Sodium | 0.200 | — |
| hydrogenphosphate | | |
| Sodium dihydrogen | 0.015 | — |
| phosphate | | |
| Sodium hydrogen | — | 0.2 |
| carbonate | | |
| Hydroxypropyl | 0.020 | — |
| methylcellulose | | |
| Sodium | — | 0.002 |
| ethylenediaminetetra | | |
| acetic acid | | |
| 1-menthol | — | 0.01 |
| Polysorbate 80 | 0.020 | — |
| Poloxamer 407 | 0.100 | — |
| Polyoxyethylene | — | 3 |
| hydrogenated castor | | |
| oil | | |
| Polyhexamethylene | 0.001 | — |
| biguanide | | |
| hydrochloride | | |
| Hydrochloric acid | q.s. | q.s. |
| Sodium hydroxide | q.s. | q.s. |
| Purified water | q.s. | q.s. |
| pH | 7.2 | 7.5 |

TABLE 5

| | Formulation | |
|---|---|---|
| | Example 10 | Formulation |
| Component | Hemorrhoidal | Example 11 |
| (Unit: g/100 mL) | agent | Vaginal cream |
| Alginic acid of | 1 | 0.5 |
| Example 1 | | |
| Ethyl aminobenzoate | 2.5 | — |
| Lidocaine | 2.25 | — |
| Hydrocortisone | 0.25 | — |
| acetate | | |
| Zinc oxide | 5 | — |
| Isopropylmethylphenol | 0.1 | — |
| Isoconazole nitrate | — | 1 |
| l-menthol | 0.25 | — |
| Petrolatum | q.s. | 8 |
| liquid paraffin | — | 8 |
| Gel carbon hydride | 30 | — |
| Glyceryl monostearate | 2.5 | — |
| Sorbitan monostearate | — | 1 |
| Polysorbate 60 | — | 3.5 |
| Behenyl alcohol | — | 5 |
| Paraben | 0.1 | — |
| Purified water | — | q.s. |

### BRIEF DESCRIPTION OF DRAWINGS

FIG. 1 is a graph that represents results of tests for the cytotoxicity of alginic acid (Example 1 and Comparative Examples 1-2).

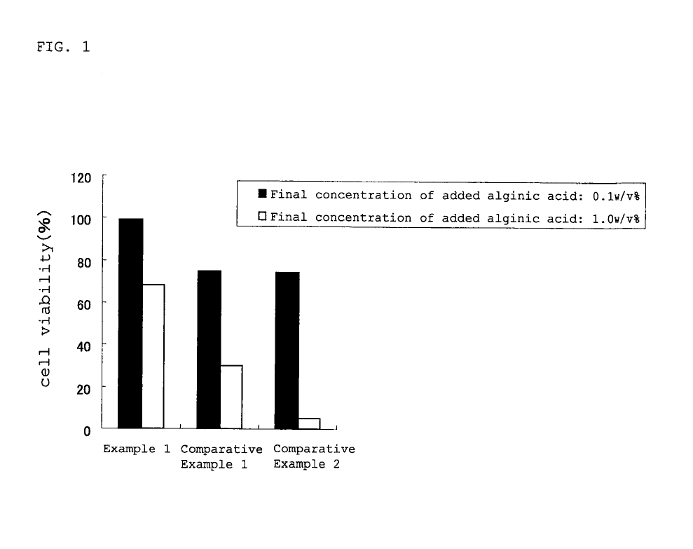

1. A composition for application to skin or mucosa that contains alginic acid and/or salts thereof comprising substantially no low-molecular-weight fraction having a molecular weight of 3,500 or less.

2. The composition for application to skin or mucosa according to claim 1, wherein the content of the fraction having a molecular weight of 380 or more to 3,500 or less is 2.5% or less based on a total amount of a fraction having a molecular weight of 380 or more of the alginic acid and/or salts thereof.

3. The composition for application to skin or mucosa according to claim 1, wherein the alginic acid and/or salts thereof is contained in an amount of 0.001 to 20% by weight based on a total weight of the composition.

4. The composition for application to skin or mucosa according to claim 1, which is a liquid formulation.

5. The composition for application to skin or mucosa according to claim 1, which is an ophthalmic composition, otologic composition, oral composition, rectal composition, vaginal composition, or urethral composition.

6. The composition for application to skin or mucosa according to claim 1, which is an eye drop or eye wash.

7. A method for reducing the cytotoxicity of alginic acid and/or salts thereof, which comprises the step of substantially removing a low-molecular-weight fraction having a molecular weight of 3,500 or less from the alginic acid and/or salts thereof.

8. Use of alginic acid and/or salts thereof that comprises substantially no low-molecular-weight fraction having a molecular weight of 3,500 or less for the manufacture of a composition for application to skin or mucosa.

9. Use of alginic acid and/or salts thereof that comprises substantially no low-molecular-weight fraction having a molecular weight of 3,500 or less for avoiding the induction of cytotoxicity in a composition for application to skin or mucosa.

10. Use of alginic acid and/or salts thereof that comprises substantially no low-molecular-weight fraction having a molecular weight of 3,500 or less for avoiding the induction of cytotoxicity on skin or mucosa, and increasing the retention of an active ingredient contained in a composition for application to skin or mucosa on skin or mucosa.

11. Alginic acid and/or salts thereof that comprises substantially no low-molecular-weight fraction having a molecular weight of 3,500 or less for avoiding the induction of cytotoxicity on skin or mucosa.

12. Alginic acid and/or salts thereof that comprises substantially no low-molecular-weight fraction having a molecular weight of 3,500 or less for avoiding the induction of cytotoxicity on skin or mucosa, and increasing the retention of an active ingredient contained in a composition for application to skin or mucosa on skin or mucosa.
